(19) United States
(12) Patent Application Publication
(10) Pub. No.: US 2008/0311226 A1
(43) Pub. Date: Dec. 18, 2008

(54) Methods and Compositions For Improving Stool Quality

(75) Inventors: Ryan Michael Yamka, Topeka, KS (US); Lauren Jay Kats, Topeka, KS (US)

Correspondence Address: COLGATE-PALMOLIVE COMPANY, 909 RIVER ROAD, PISCATAWAY, NJ, 08855, US

(73) Assignee: HILL'S PET NUTRITION, INC., Topeka, KS (US)

(21) Appl. No.: 12/091,949

(22) PCT Filed: Nov. 3, 2006
(86) PCT No.: PCT/US06/60520

§ 371 Date: Jul. 30, 2008

Related U.S. Application Data
(60) Provisional application No. 60/733,905, filed on Nov. 4, 2005.

(51) Int. Cl. A61K 33/06

(52) U.S. Cl. 424/684

(57) ABSTRACT

A method for improving stool quality by administering to an animal a composition comprising from about 1% to about 20% zeolite. Preferably, the zeolite is fed to ah animal as part of a food composition containing least one ingredient suitable for consumption by an animal.

### CROSS-REFERENCE TO RELATED APPLICATIONS

This application claims priority to U.S. Provisional Application Ser. No. 60/733,905 filed Nov. 4, 2005, the disclosure of which is incorporated herein by reference.

### BACKGROUND OF THE INVENTION

1. Field of the Invention

This invention relates generally to methods for improving stool quality and particularly to methods for using zeolites to improve stool quality.

2. Description of the Prior Art

Stool quality is generally determined by five factors: food ingredient digestibility, fiber level, health status, activity level, and water intake. When these factors are balanced, stools are generally formed, firm, dark, and exhibit a relatively reduced odor. Stools exhibiting these properties are considered good quality stools. If the factors are not balanced, stools are generally soft, loose, watery, light-colored, and exhibit a relatively increased odor. Stools exhibiting these properties, particularly loose, watery stools, are considered poor quality stools.

Poor stool quality can be caused by various factors, e.g., abnormal intestinal motility, increases in intestinal permeability, the presence of nonabsorbable osmotically active substances in the intestine, or agents that cause diarrhea. Similarly, some animal foods, particularly those known in the art as chunk and gravy animal foods, can cause poor stool quality. Often, an animal consuming such foods has a fecal discharge that is irregular and undesirable. Such discharge is generally characterized by loose, watery stools. In some instances, the discharge may be classified as diarrhea.

Methods for managing poor stool quality have focused on the use of drugs that combat diarrhea and on compositions that affect stool quality. For example, U.S. Pat. No. 6,280,779 discloses foods containing chemically modified starches and gums useful for maintaining normal bowel health and promoting production of acceptable stool quality; WO05063271A1 discloses the use of herbal compositions to treat gastro-intestinal, disorders and affect stool quality; U.S. Pat. No. 5,919,760 discloses the use of octreotide to treat diarrhea; and WO9625940A1 discloses the use of compositions comprising carrots, rice, bananas and glucose for treating diarrhea. While these methods are useful, there continues to be a need for new methods and compositions for improving stool quality.

### SUMMARY OF THE INVENTION

It is, therefore, an object of the present invention to provide methods for improving stool quality.

It is another object of the present invention to provide compositions useful for improving stool quality.

It is a further object of the invention to provide articles of manufacture in the form of kits that contain combinations of compositions useful for improving stool quality.

These and other objects are achieved using novel methods for improving stool quality comprising administering to an animal a composition comprising a stool quality improving amount of zeolite. Generally, the composition is a food composition comprising at least about 1% zeolite and at least one ingredient selected from the group consisting of protein, fat, carbohydrate, fiber, and combinations thereof, most preferably in amounts of from about 1% to about 20%. Kits comprising compositions for animal consumption and zeolite are also provided. Compositions for animal consumption containing zeolite are surprisingly effective for improving stool quality.

Other and further objects, features and advantages of the present invention will be readily apparent to those skilled in the art.

### DETAILED DESCRIPTION OF THE INVENTION

### Definitions

The term “zeolite” means a single zeolite or a mixture of two or more zeolites.

The term “animal” means any animal susceptible to or suffering from poor stool quality. An animal is “susceptible to” a disease or condition if the animal exhibits symptoms that indicate that the animal is likely to develop the condition or disease. An animal is “suffering from” a disease or condition if the animal exhibits symptoms that are indicative that the animal has developed the condition or disease.

The term “in conjunction” means that one or more of the compositions or compounds (e.g., anti-diarrhea drugs or composition components) of the present invention are administered to an animal (1) together in a food composition or (2) separately at the same or different frequency using the same or different administration routes at about the same time or periodically. “Periodically” means that the compositions; food compositions, and compounds are administered on a dosage schedule acceptable for a specific composition, food composition, and compound and that the food compositions are administered or fed to an animal routinely as appropriate for the particular animal. “About the same time” generally means that the compositions, composition components, anti-diarrhea drugs, and food compositions are administered at the same time or within about 72 hours of each other. In conjunction specifically includes administration schemes wherein anti-diarrhea drugs are administered for a prescribed period and the compositions are administered indefinitely.

The term “anti-diarrhea drug” means any compound, composition, or drug useful for preventing or treating diarrhea.

The term “single package” means that the components of a kit are physically associated in or with one or more containers and considered a unit for manufacture, distribution, sale, or use. Containers include, but are not limited to, bags, boxes, bottles, shrink wrap packages, stapled or otherwise affixed components, or combinations thereof. A single package may be containers of individual food compositions physically associated such that they are considered a unit for manufacture, distribution, sale, or use.

The term “virtual package” means that the components of a kit are associated by directions on one or more physical or virtual kit components instructing the user how to obtain the other components, e.g., in a bag containing one component and directions instructing the user to go to a website, contact a recorded message, view a visual message, or contact a caregiver or instructor to obtain instructions on how to use the kit.

This invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise, e.g., reference to “a zeolite” includes a plurality of such zeolites.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents, patent applications, and publications mentioned herein are incorporated herein by reference to the extent allowed by law for the purpose of describing and disclosing the compounds and methodologies reported therein that might be used with the present invention. However, nothing herein is to be construed as an admission that the invention is not entitled to antedate such disclosure by virtue of prior invention.

### THE INVENTION

In one aspect, the present invention provides methods for improving stool quality. The methods comprise administering to an animal a composition comprising a stool quality improving amount of zeolite. In preferred embodiments, the composition comprises one or more ingredients suitable for consumption by an animal and zeolite in amounts of at least about 1% by weight, preferably in amounts of from about 1% to about 20%, most preferably in amounts of from about 1% to about 10%. The invention is based upon the novel discovery that zeolite will improve stool quality when consumed by an animal.

The compositions are administered orally using any suitable form for oral administration, e.g., tablets, pills, suspensions, solutions (possibly admixed with drinking water), emulsions, capsules, powders, syrups, and palatable feed compositions (a confectionery for a human or a treat or flavored treat for an animal). In a preferred embodiment, the zeolite and other ingredients, are admixed during manufacture process used to prepare the composition.

In one embodiment, the method comprises administering the composition in conjunction with one or more gastrointestinal tract improving agents selected from the group consisting of probiotics and prebiotics. In another, the method comprises administering the composition in conjunction with one or more anti-diarrhea drugs.

In another aspect, the present invention provides compositions for animal consumption that improve stool quality. The compositions comprise a stool quality improving amount of zeolite and at least one ingredient suitable for consumption by ah animal. Preferably, the compositions comprise at least about 1% by weight zeolite and an ingredient is selected from the group consisting of protein, fat, carbohydrate, and fiber. Preferably, the compositions contain zeolite in amounts of from about 1% to about 20%, most preferably in amounts of from about 1% to about 10% or from about 2% to about 6%. The zeolite should be present at concentrations that are not toxic or otherwise deleterious to an animal's health. Thus, the zeolite should be present at concentrations that do not cause undesirable effects on digestion, particularly long term undesirable effects on digestion, such as undesirable effects lasting several days or longer. Undesirable effects on digestion may include constipation or diarrhea.

The zeolite may be incorporated into the composition during the processing of the formulation, such as during and/or after mixing of other components of the composition. Distribution of these components into the composition is accomplished by conventional means. Unless otherwise specifically indicated, all weights and concentrations for the compositions of the present invention are based on dry weight of a composition after all components and ingredients are admixed.

Zeolites are three-dimensional, microporous, crystalline solids with a well-defined structure comprising aluminum, silicon, and oxygen in a tetrahedral framework. Zeolites have large open spaces or cages in their structure that form channels allowing, easy movement of ions and molecules into and out of the structure. The negative charge of the Al is balanced by the presence of cations including calcium, magnesium, sodium, potassium, and iron.

Nearly 50 naturally occurring members of the zeolite family have been discovered and more than 150 zeolite: structural types have been synthesized. Except where the context demands otherwise, the term “zeolite” is used generically herein to encompass any mineral including but not limited to: the Analcime Family including analcime (hydrated sodium aluminum silicate), pollucite (hydrated cesium sodium aluminum silicate), wairakite (hydrated calcium sodium aluminum silicate), bellbergite (hydrated potassium barium strontium sodium aluminum silicate), bikitaite (hydrated lithium aluminum silicate), boggsite (hydrated calcium sodium aluminum silicate) and brewsterite (hydrated strontium barium sodium calcium aluminum silicate); the Chabazite Family including chabazite (hydrated calcium aluminum silicate), willhendersonite (hydrated potassium calcium aluminum silicate), cowlesite (hydrated calcium aluminum silicate), dachiardite (hydrated calcium sodium potassium aluminum silicate), edingtonite (hydrated barium calcium aluminum silicate), epistilbite (hydrated calcium aluminum silicate), erionite (hydrated sodium potassium calcium aluminum silicate), faujasite (hydrated sodium calcium magnesium aluminum silicate) and ferrierite (hydrated sodium potassium magnesium calcium aluminum silicate); the Gismondine Family including amicite (hydrated potassium sodium aluminum silicate), garronite (hydrated calcium aluminum silicate), gismondine (hydrated barium calcium aluminum silicate), gobbinsite (hydrated sodium potassium calcium aluminum silicate), gmelinite (hydrated sodium calcium aluminum silicate), gonnardite (hydrated sodium calcium aluminum silicate) and goosecreekite (hydrated calcium aluminum silicate); the Harmotome Family including harmotome (hydrated barium potassium aluminum silicate), phillipsite (hydrated potassium sodium calcium aluminum silicate) and wellsite (hydrated barium calcium potassium aluminum silicate); the Heulandite Family including clinoptilolite (hydrated sodium potassium calcium aluminum, silicate), heulandite (hydrated sodium calcium aluminum silicate), laumontite (hydrated calcium aluminum silicate), levyne (hydrated calcium sodium potassium aluminum silicate), mazzite (hydrated potassium sodium, magnesium calcium aluminum silicate), merlinoite (hydrated potassium sodium calcium barium aluminum silicate), montesommaite (hydrated potassium sodium aluminum silicate) and mordenite (hydrated sodium potassium calcium aluminum silicate); the Natrolite Family including mesolite (hydrated sodium calcium aluminum silicate), natrolite (hydrated sodium aluminum silicate), scolecite (hydrated calcium aluminum silicate), offretite (hydrated calcium potassium magnesium aluminum silicate), paranatrolite (hydrated sodium aluminum silicate), paulingite (hydrated potassium calcium sodium barium aluminum silicate) and perlialite (hydrated, potassium sodium calcium strontium aluminum silicate); and the Stilbite Family including barrerite (hydrated sodium potassium calcium aluminum silicate), stilbite (hydrated sodium calcium aluminum silicate), stellerite (hydrated calcium aluminum silicate), thomsonite (hydrated sodium calcium aluminum silicate), tschemichite (hydrated calcium aluminum silicate) and yugawaralite (hydrated calcium aluminum silicate). The term zeolite is also used generically herein to encompass any mineral including the natural zeolite, synthetic materials, silicate minerals and phosphate minerals that have a zeolite like structure. Synthetic zeolite include zeolite A, non-fibrous zeolite, zeolite 4A, zeolite MS4A, and zeolite MS5A. Zeolite silicate minerals include hsianghualite, lovdarite, viseite, partheite, prehnite, roggianite, apophyllite, gyrolite, maricopaite, okenite, tacharanite and tobermorite. Zeolite phosphate minerals include kehoeite, pahasapaite and tiptopite.

Any type or form of zeolite that is acceptable for use in a composition for animal consumption may be used in the present invention. An illustrative example of a zeolite suitable for use in the present invention is clinoptilolite. Clinoptilolite is commercially available as SAFETYSORB from Safetysorb Zeolite, Inc. of Las Vegas, Nev. but may be obtained from any source.

This invention provides a variety of compositions containing zeolite, e.g., foods, nutritional diets, supplements, treats, and toys such as chewable and consumable toys.

In some embodiments, the composition is a food. Both liquid and solid foods are provided. When the food is a liquid, the zeolite may be admixed with the food. Where the food is solid, the zeolite may be coated on the food, incorporated into the food, or both. The food includes both dry foods and wet foods. The non-zeolite components of the food and their typical proportions are known to skilled artisans and typically include carbohydrates, proteins, fats, fibers, and/or nutritional ingredients such as vitamins, minerals, and the like.

Specific suitable amounts for each component in a composition will depend on a variety of factors such as the species of animal consuming the composition; the particular components included in the composition; the age, weight, general health, sex, and diet of the animal; the animal's consumption rate; and the like.

The protein food ingredient is obtained from a variety sources such as plants, animals, or both. Animal protein includes meat, meat by-products, dairy, and eggs. Meats include the flesh from poultry, fish, and animals such as cattle, swine, sheep, goats, and the like. Meat by-products include lungs, kidneys, brain, livers, stomachs, and intestines. The protein food ingredient may also be free amino acids and/or peptides. Preferably, the protein food ingredient comprises meat, a meat by-product, dairy products, or eggs. The fat and carbohydrate food ingredient is obtained from a variety of sources such as animal fat, fish oil, vegetable oil, meat, meat by-products, grains, other animal or plant sources, and mixtures thereof. Grains include wheat, corn, barley, and rice. The fiber food ingredient is obtained from a variety of sources such as vegetable fiber sources, e.g., cellulose, beet pulp, peanut hulls, and soy fiber.

Supplements useful in the present invention include a feed used with another feed to improve the nutritive balance or performance of the total. Supplements include compositions that are fed undiluted as a supplement to other feeds, offered free choice with other parts of an animal's ration that are separately available, or diluted and mixed with an animal's regular feed to produce a complete feed. The AAFCO provides a discussion relating to supplements in the American Feed Control Officials, Inc. Official Publication, page 220 (2003). Supplements may be in various forms including powders, liquids, syrups, pills, encapsulated compositions, and the like.

Treats include compositions that are given to an animal to entice the animal to eat during a non-meal time, e.g., dog bones for canines. Treats may be nutritional wherein the composition comprises one or more nutrients, and may have a composition as described above for food. Non-nutritional treats encompass: any other treats that are non-toxic. The zeolite is coated onto the treat, incorporated into the treat, or both.

Toys include chewable toys such as artificial bones. The zeolite can form a coating on the surface of the toy or on the surface of a component of the toy, be incorporated partially or fully throughout the toy, or both. In one embodiment, the zeolite is orally accessible by the intended user. There are a wide range of suitable toys currently marketed, e.g., U.S. Pat. No. 5,339,771, U.S. Pat. No. 5,419,283, and references disclosed therein. This invention provides both partially consumable toys, e.g., toys comprising plastic components, and fully consumable toys, e.g., rawhides and various artificial bones. Further, the invention provides toys for both human and non-human use, particularly for companion, farm, and zoo animal use; and particularly for dog, cat, or bird use.

The compositions may contain additional ingredients such as vitamins, minerals, fillers, palatability enhancers, binding agents, flavors, stabilizers, emulsifiers, sweeteners, colorants, buffers, salts, coatings, and the like known to skilled artisans. Stabilizers include substances that tend to increase the shelf life of the composition such as preservatives, synergists and sequestrants, packaging gases, stabilizers, emulsifiers, thickeners, gelling agents, and humectants. Examples of emulsifiers and/or thickening agents include gelatin, cellulose ethers, starch, starch esters, starch ethers, and modified starches. Specific amounts for each composition component, food ingredient, and other ingredients will depend on a variety of factors such as the particular components and ingredients included in the composition; the species of animal; the animal's age, body weight, general health, sex, and diet; the animal's consumption rate; the type of disease being treated (if any); and the like. Therefore, the ingredient amounts may vary widely and may deviate from the preferred proportions described herein. The amount of such additives in a composition typically is up to about 5% by weight.

Particularly when the composition is an animal food, vitamins and minerals preferably are included in amounts required to avoid deficiency and maintain health. These amounts are readily available in the art. The National Research Council (NRC) provides recommended amounts of such ingredients for farm animals. See, e.g., Nutrient Requirements of Swine (10th Rev. Ed., Nat'l Academy Press, Wash. D.C., 1998), Nutrient Requirements of Poultry (9th Rev. Ed., Nat'l Academy Press, Wash. D.C., 1994), Nutrient Requirements of Horses (5th Rev. Ed., Nat'l Academy Press, Wash. D.C., 1989), etc. The American Feed Control Officials (AAFCO) provides recommended amounts of such ingredients for dogs and cats. See American Feed Control Officials, Inc., Official publication, pages 126-140 (2003). Vitamins generally useful as food additives include vitamin A, vitamin B1, vitamin B2, vitamin B6, vitamin B12, vitamin C, vitamin D, vitamin E, vitamin H (biotin), vitamin K, folic acid, inositol, niacin, and pantothenic acid. Minerals and trace elements generally useful as food additives include calcium, phosphorus, sodium, potassium, magnesium, copper, zinc, choline, and iron. The compositions may contain additional ingredients intended to maintain or improve the health of the animal, e.g., medications, herbs, holistic drugs and compositions, and the like:

Compositions of the present invention, particularly foods, can be prepared in a dry form using conventional processes. In one embodiment, dry ingredients, including animal protein sources, plant protein sources, grains, etc. are ground and mixed together, Moist or liquid ingredients, including fats, oils, animal protein sources, water, etc. are then added to and mixed with the dry mix. The mixture is then processed into kibbles or similar dry pieces. Kibble is often formed using an extrusion process in which the mixture of dry and wet ingredients is subjected to mechanical work at a high pressure and temperature, and forced through small openings and cut off into kibble by a rotating knife. The wet kibble is then dried and optionally coated with one or more topical coatings which may include flavors, fats, oils, powders, and the like. Kibble also can be made from the dough using a baking process, rather than extrusion, wherein the dough is placed into a mold before dry-heat processing.

The zeolite may be added to the food composition in its normal preparation procedure such as mixing, extrusion, baking and the like or is preferably added after its preparation post extrusion, such as by spraying or coating the surface of the food. This is particularly desirable for dry foods wherein the extruded strands are contacted with the zeolite (or a solution comprising the zeolite) by spraying or coating the extruded strands before the strands are cut into a kibble, or the kibble is contacted with the zeolite (or a solution comprising the zeolite) by spraying, coating or dipping the kibble per se.

For topical application to a food, the zeolite is mixed with a carrier composition to facilitate application to the surface of the food, composition. For example, a liquid, slurry, light gel, or watery solid can all be utilized as a carrier for the compound(s) of this composition. A standard spraying or dipping apparatus is employed to apply the compound(s) to the surface of the food composition. An example of such a carrier is a minced animal by-product treated with proteases in conjunction with amino acids, reducing sugar(s) and thiamin. The carrier is then mixed with the zeolite and coated onto a kibble, thereby preparing a very palatable and acceptable dry food. In a certain preferred embodiment, the zeolite may simply be mixed with a commercial liquid palatant enhancer or other flavor composition to create a novel flavor palatant which can then be topically applied to the composition. Suitable commercial liquid palatant enhancers for use with the zeolite in the present invention include any known or commercially available liquid palatant enhancers commercially available from pet food palatant enhancer or other flavor suppliers known to those of skill in the art.

Compositions of the present invention, particularly foods, can; be prepared in a canned or wet form using conventional pet food processes. In one embodiment, ground animal (e.g., mammal, poultry, fish and/or seafood) proteinaceous tissues are mixed with the other ingredients, including fish oils, cereal grains, other nutritionally balancing ingredients, special purpose additives (e.g., vitamin and mineral mixtures, inorganic salts, cellulose and beet pulp, bulking agents, and the like). Water sufficient for processing may also be added. The wet form ingredients are typically mixed in a vessel suitable for heating while blending the components. Heating of the mixture may be accomplished using any suitable manner, such as by direct steam injection or by using a vessel fitted with a heat exchanger. Following the addition of the last ingredient, the mixture is heated to a temperature range of from about 50° F. (10° C.) to about 212° F. (100° C.). Temperatures outside this range are acceptable, but may be commercially impractical without use of other processing aids. When heated to the appropriate temperature, the material will typically be in the form of a thick liquid. The thick liquid is filled into cans. A lid is applied, and the container is hermetically sealed. The sealed can is then placed into conventional equipment designed to sterilize the contents. This is usually accomplished by heating to temperatures of greater than about 230° F. (110° C.) for an appropriate time, which is dependent on the temperature used and the composition.

For wet foods, the zeolite can be incorporated into the wet food composition along with a carrier such as an alcohol composition (i.e., propylene glycol or dipropylene glycol), a cyclodextrin, a maltodextrin, or a starch. Alternatively, the zeolite can be mixed into the dry materials before forming the wet food composition.

Treats of the present invention can be prepared by an extrusion or baking process similar to those described above for dry food. Other processes also may be used to either coat the flavoring composition on the exterior of existing treat forms, or inject it into an existing treat form. Animal toys of the present invention are typically prepared by coating any existing toy with a flavoring composition having the zeolite mixed therein.

In one embodiment, composition further comprises one or more gastrointestinal tract improving agents selected from the group consisting of probiotics and prebiotics. In another, the composition further comprises one or more anti-diarrhea drugs.

The compositions and methods of this invention are useful for a variety of human and non-human animals, including avian, bovine, canine, equine, feline, hicrine, murine, ovine, and porcine animals. Preferably, the animal is a canine or feline.

In a further aspect, the present invention provides kits suitable for administering a composition for consumption by an animal comprising a stool quality improving amount of zeolite and at least one ingredient suitable for consumption by an animal. The kits comprise in separate containers in a single package or in separate containers in a virtual package, as appropriate for the kit component, a stool quality improving amount of zeolite and at least one of (1) one or more ingredients suitable for consumption by ah animal, (2) instructions for how to combine the zeolite and the ingredient(s) to produce a composition for animal consumption that improves stool quality, (3) instructions for how to use the composition of the present invention to improve stool quality, particularly for the benefit of the animal, (4) one or more gastrointestinal tract improving agents selected from the group consisting of probiotics and prebiotics, and (5) one or more anti-diarrhea drugs. When the kit comprises a virtual package, the kit is limited to instructions in a virtual environment in combination with one or more physical kit components.

The kit contains the zeolite and other components in amounts sufficient to improve stool quality. Generally, kits contain the zeolite in amounts sufficient to produce a composition having at least about 1% zeolite, most preferably in amounts of from about 1% to about 20%, and at least one ingredient selected from the group consisting of protein, fat, carbohydrate, fiber, and combinations thereof. Typically, the zeolite and the ingredient(s) are admixed just before consumption by an animal. In one embodiment, the kit contains a packet of zeolite and a container of food for consumption by an animal. The kit may contain additional items such as a device for mixing the zeolite and ingredient(s) or a device for containing the admixture, e.g., a food bowl. In another embodiment, the zeolite is mixed with additional nutritional supplements such as vitamins and minerals that promote-good health in an animal.

In another aspect, the present invention provides a means for communicating information about or instructions for one or more of (1) using zeolite, to improve stool quality, (2) admixing zeolite with the other components of the present invention, (3) administering zeolite to an animal, alone or in combination with the other elements of the present invention, and (4) using the kits of the present invention for improving stool quality. The communicating means comprises a document, digital storage media, optical storage media, audio presentation, or visual display containing the information or instructions. Preferably, the communication is a displayed web site or a brochure, product label, package insert, advertisement, or visual display containing such information or instructions. Useful information includes one or more of (1) methods and techniques for combining and administering the zeolite and ingredients and (2) contact information for consumers to use if they have a question about the invention and its use. Useful instructions include amounts for mixing and administration amounts and frequency. The communication means is useful for instructing on the benefits of using the present invention and communicating the approved methods for administering the invention to an animal.

In a further aspect, the present invention provides compositions for animal consumption that improve stool quality and have a beneficial effect on the gastrointestinal tract. The compositions comprise a stool quality improving amount of zeolite, at least one ingredient suitable for consumption by an animal, and a gastrointestinal tract improving agent selected from the group consisting of probiotics and prebiotics. Probiotics are live microorganisms that have a beneficial effect in the prevention and treatment of specific medical conditions when ingested. Probiotics are believed to exert biological effects through a phenomenon known as colonization resistance. The probiotics facilitate a process whereby the indigenous anaerobic flora limits the concentration of potentially harmful (mostly aerobic) bacteria in the digestive tract. Other modes of action, such as supplying enzymes or influencing enzyme activity in the gastrointestinal tract, may also account for some of the other functions that have been attributed to probiotics. Prebiotics are nondigestible food ingredients that beneficially affect host health by selectively stimulating the growth and/or activity of bacteria in the colon. The prebiotic, fructooligosaccharide (FOS) is found naturally in many foods such as wheat, onions, bananas, honey, garlic, and leeks. FOS can also be isolated from chicory root or synthesized enzymatically from sucrose. FOS fermentation in the colon results in a large number of physiologic effects including increasing the numbers of bifidobacteria in the colon, increasing calcium absorption, increasing fecal weight, shortening of gastrointestinal transit time, and possibly lowering blood lipid levels. The increase in bifidobacteria has been assumed to benefit human health by producing compounds to inhibit potential pathogens, by reducing blood ammonia levels, and by producing vitamins and digestive enzymes. Probiotic bacteria such as Lactobacilli of Bifidobacteria are believed to positively affect the immune response by improving the intestinal microbial balance leading to enhanced antibody production and phagocytic (devouring or killing) activity of white blood cells. Bifidobacterium lactis could be an effective probiotic dietary supplement for enhancing some aspects of cellular immunity in the elderly. Probiotics enhance systemic cellular immune responses and may be useful as a dietary supplement to boost natural immunity in otherwise healthy adults. Probiotics include many types of bacteria but generally are selected from four genera of bacteria: Lactobacillus acidpphillus, Bifidobacteria, Lactococcus, and Pediococcus. The amount of probiotics and prebiotics to be administered to the animal is determined by the skilled artisan based upon the type and nature of the probiotic and prebiotic and the type and nature of the animal, e.g., the age, weight; general health, sex, extent of microbial depletion, presence of harmful bacteria, and diet of the animal. Generally, probiotics are administered to the animal in amounts of from about one to about twenty billion colony forming units (CPUs) per day for the healthy maintenance of intestinal microflora, preferably from about 5 billion to about 10 billion live bacteria per day. Generally, prebiotics are administered in amounts sufficient to positively stimulate the healthy microflora in the gut and cause these “good” bacteria to reproduce. Typical amounts are from about one to about 10 grams per serving or from about 5 percent to about 40 percent of the recommended daily dietary fiber for an animal. The probiotics and prebiotics can be made part of the composition by any suitable means. Generally, the agents are mixed with the composition or applied to the surface of the composition, e.g., by sprinkling. When the agents are part of a kit, the agents can be admixed with other materials or in their own package.

In another aspect, the present invention provides methods for manufacturing compositions for animal consumption that improve stool, quality. The methods comprise admixing a stool quality improving amount of zeolite and at least one ingredient suitable for consumption by an animal. The zeolite is added to the composition is amounts of at least about 1% by weight zeolite, preferably in amounts of from about 1% to about 20%, most preferably in amounts of from about 1% to about 10%.

The compositions, methods, and kits are useful for improving stool quality.

### EXAMPLES

This invention can be further illustrated by the following examples although it will be understood that these examples are included merely for purposes of illustration and are not intended to limit the scope of the invention unless otherwise; specifically indicated.

### Example 1

Four stool quality studies were performed on the two groups of dogs (groups 1 and 2) utilizing foods with or without zeolite. Each study was designed to determine, the quality of stool produced by the dog when they were fed one of the two foods. Each study consisted of ten dogs being fed the food for a period of seven days. All foods were fed at maintenance level. The ingredient and nutrient compositions of the foods fed to group 1 are; listed in Tables 1 and 2. The foods fed to group 2 are listed in Tables 5 and 6. The results are shown in Tables 3 and 4 and in Tables 7 and 8.

TABLE 1
Ingredient Composition Of Foods Fed To Group 1.

| Ingredient, % | Treatment A | Treatment B |
|---|---|---|
| Corn Starch | 55.736 | 57.700 |
| Poultry Liver Hydrolysate | 29.336 | 29.303 |
| Soybean Oil | 4.050 | 4.050 |
| Cellulose | 4.000 | 4.000 |
| Zeolite | 2.000 | 0.000 |
| Calcium Carbonate | 1.140 | 0.950 |
| Dicalcium Phosphate | 1.000 | 1.120 |
| Glyceryl Monostearate | 0.750 | 0.750 |
| Sodium Bicarbonate | 0.650 | 0.000 |
| Potassium Chloride | 0.620 | 0.700 |
| Salt Iodized | 0.000 | 0.450 |
| Choline Chloride | 0.300 | 0.450 |
| DL-Methionine | 0.200 | 0.300 |
| Vitamin Premix | 0.120 | 0.120 |
| Mineral Premix | 0.075 | 0.075 |
| Taurine | 0.020 | 0.020 |
| Ethoxyquin | 0.013 | 0.013 |

TABLE 2
Nutrient Composition Of Foods Fed To Group 1,

| Nutrient^{a} | Treatment A | Treatment B |
|---|---|---|
| Protein, % | 19.08 | 19.26 |
| Fat, % | 15.92 | 15.26 |
| Ash, % | 4.38 | 5.82 |
| Crude Fiber, % | 2.13 | 2.75 |
| Calcium, % | 0.63 | 0.68 |
| Phosphorous, % | 0.54 | 0.52 |
| Potassium, % | 0.66 | 0.59 |
| Sodium, % | 0.29 | 0.31 |
| Sulfur, % | 0.33 | 0.36 |
| Chloride, % | 0.81 | 0.54 |
| Magnesium, % | 0.05 | 0.05 |
| Base Excess^{b}, meq | −97.7 | −12.9 |

^{a}Dry Matter basis
^{b}Base Excess = (Sodium + Potassium + Calcium + Magnesium) − Chloride + Sulfur + Phosphorous)

TABLE 3
Stool Scores And Food Intake Of Group 1 Fed Treatment A (With
Zeolite)

| | | Standard | | | |
|---|---|---|---|---|---|
| | Mean | Deviation | Minimum | Maximum | Count |
| Food Intake | 261.2 | 46.1 | 117 | 326 | 69 |
| Stool | 4.7 | 0.8 | 1.0 | 5.0 | 71 |

TABLE 4
Stool Scores And Food Intake Of Group 1 Fed Treatment B (No Zeolite)

| | | Standard | | | |
|---|---|---|---|---|---|
| | Mean | Deviation | Minimum | Maximum | Count |
| Food Intake | 209.3 | 46.2 | 100 | 302 | 70 |
| Stool | 4.3 | 1.3 | 1.0 | 5.0 | 73 |

TABLE 5
Ingredient Composition Of Foods Fed To Group 2.

| Ingredient, % | Treatment C | Treatment D |
|---|---|---|
| Corn Starch | 55.978 | 57.613 |
| Poultry Liver | 28.155 | 28.776 |
| Hydrolysate | | |
| Soybean Oil | 5.500 | 4.663 |
| Cellulose | 4.000 | 4.000 |
| Zeolite | 2.000 | 0.000 |
| Calcium Carbonate | 1.220 | 0.950 |
| Dicalcium Phosphate | 0.840 | 1.120 |
| Glyceryl Monostearate | 0.750 | 0.750 |
| Potassium Chloride | 0.570 | 0.700 |
| Salt Iodized | 0.270 | 0.450 |
| Choline Chloride | 0.300 | 0.450 |
| DL-Methionine | 0.200 | 0.300 |
| Vitamin Premix | 0.120 | 0.120 |
| Mineral Premix | 0.075 | 0.075 |
| Taurine | 0.010 | 0.020 |
| Ethoxyquin | 0.013 | 0.013 |

TABLE 6
Nutrient Composition Of Foods Fed To Group 2.

| Nutrient^{a} | Treatment C | Treatment D |
|---|---|---|
| Protein, % | 21.65 | 20.31 |
| Fat, % | 15.81 | 13.86 |
| Ash, % | 6.40 | 4.79 |
| Crude Fiber, % | 3.62 | 3.09 |
| Calcium, % | 0.75 | 0.68 |
| Phosphorous, % | 0.50 | 0.58 |
| Potassium, % | 0.76 | 0.75 |
| Sodium, % | 0.33 | 0.33 |
| Sulfur, % | 0.35 | 0.37 |
| Chloride, % | 0.90 | 0.85 |
| Magnesium, % | 0.06 | 0.05 |
| Base Excess^{b}, meq | −1.2 | −91.8 |

^{a}Dry Matter basis
^{b}Base Excess = (Sodium + Potassium + Calcium + Magnesium) − Chloride + Sulfur + Phosphorous)

TABLE 7
Stool Scores And Food Intake Of Group 2 Fed Treatment C (With Zeolite)

| | | Standard | | | |
|---|---|---|---|---|---|
| | Mean | Deviation | Minimum | Maximum | Count |
| Food Intake | 189.8 | 40.5 | 39 | 251 | 70 |
| Stool | 3.9 | 1.0 | 2 | 5 | 93 |

TABLE 8
Stool Scores And Food Intake Of Group 2 Fed Treatment D (No Zeolite)

| | | Standard | | | |
|---|---|---|---|---|---|
| | Mean | Deviation | Minimum | Maximum | Count |
| Food Intake | 184.6 | 24.5 | 0 | 206 | 70 |
| Stool | 3.4 | 1.0 | 1 | 5 | 81 |

Referring to the results, the data show that zeolite is effective for improving stool quality, i.e., animals consuming food compositions containing zeolite have more desirable stools than those consuming foods without zeolite.

In the specification, there have been disclosed typical preferred embodiments of the invention and, although specific terms are employed, they are used in a generic and descriptive sense only and not for purposes of limitation, the scope of the invention being set forth in the following claims. Obviously many modifications and variations of the present invention are possible in light of the above teachings. It is therefore to be understood that within the scope of the appended claims the invention may be practiced otherwise than as specifically described.

What is claimed is:

1. A method for improving stool quality comprising administering to an animal a composition comprising a stool quality improving amount of zeolite.

2. The method of claim 1 wherein the zeolite comprises at least about 1% by weight of the composition.

3. The method of claim 1 wherein the zeolite comprises from about 1% to about 20% by weight of the composition.

4. The method of claim 1 wherein the zeolite comprises a three-dimensional, microporous, crystalline solid with a well-defined structure comprising aluminum, silicon, and oxygen in a tetrahedral framework.

5. The method of in claim 1 wherein the zeolite is selected from the group consisting of analcime, pollucite, wairakite, bellbergite, bikitaite, boggsite, brewsterite, chabazite, willhendersonite, cowlesite, dachiardite, edingtoriite, epistilbite, erionite, faujasite, ferrierite, amicite, garronite, gismondine, gobbinsite, gmelinite, gonnardite, goosecreekite, harmotome, phillipsite, wellsite, clinoptilolite, heulandite, laumontite, levyne, mazzite, merlinoite, montesommaite, mordenite, mesolite, natrolite, scolecite, offretite, paranatrolite, paulingite, perlialite, barrerite, stilbite, stellerite, thomsonite, tschemichite, yugawaralite, and mixtures thereof.

6. The method of claim 1 wherein the composition is a food.

7. The method of claim 1 wherein the composition is a nutritional diet, supplement, treat, or toy.

8. The method of claim 1 wherein the animal is a canine or a feline.

9. The method of claim 1 further comprising administering me zeolite in conjunction one or more gastrointestinal tract improving agents selected from the group consisting of probiotics and prebiotics.

10. The method of claim 1 further comprising administering the zeolite in conjunction one or more anti-diarrhea drugs.

11. A composition comprising a stool quality improving, amount of zeolite and at least one ingredient suitable for consumption by an animal.

12. The composition of claim 11 wherein the zeolite comprises at least about 1% by weight of the composition.

13. The composition of claim 11 wherein the zeolite comprises from about 1% to about 20% by weight of the composition.

14. The composition of claim 11 wherein the zeolite comprises a three-dimensional, microporous, crystalline solid with a well-defined structure comprising aluminum, silicon, and oxygen in a tetrahedral framework.

15. The composition of in claim 11 wherein the zeolite is selected from the group consisting of analcime, pollucite, wairakite, bellbergite, bikitaite, boggsite, brewsterite, chabazite, willhendersonite, cowlesite, dachiardite, edingtonite, epistilbite, erionite, faujasite, ferrierite, amicite, garronite, gismondine, gobbinsite, gmelinite, gonnardite, goosecreekite, harmotome, phillipsite, wellsite, clinoptilolite, heulandite, laumontite, levyne, mazzite, merlinoite, montesommaite, mordenite, mesolite, natrolite, scolecite, offretite, paranatrolite, paulingite, perlialite, barrerite, stilbite, stellerite, thomsonite, tschemichite, yugawaralite, and mixtures thereof.

16. The composition of claim 11 wherein the ingredient is selected from the group consisting of protein, fat, carbohydrate, fiber, and combinations thereof.

17. The composition of claim 11 wherein the composition is a food, a nutritional diet, a supplement, an animal treat, or a toy.

18. The composition of claim 11 wherein the animal is a canine or a feline.

19. The composition of claim 11 further comprising one or more gastrointestinal tract improving agents selected from the group consisting of probiotics and prebiotics

20. The composition of claim 11 further comprising one or more anti-diarrhea drugs.

21. A kit comprising in separate containers in a single package or in separate containers in a virtual package, as appropriate for the kit component, a stool quality improving amount of zeolite and at least one of (1) one or more ingredients suitable for consumption by an animal, (2) instructions for how to combine the zeolite and the ingredient(s) to produce a composition for animal consumption that improves stool quality, (3) instructions for how to use the composition of the present invention to improve stool quality, (4) one or more gastrointestinal tract improving agents selected from the group consisting of probiotics and prebiotics, and (5) one or more anti-diarrhea drugs.

22. The kit of claim 21 comprising zeolite in amounts sufficient to comprise from about 1% to about 20% by weight of the composition when the zeolite is admixed with the ingredient(s).

23. A means for communicating information about or instructions for one or more of (1) using zeolite to improve stool quality, (2) admixing zeolite with the other components of the present invention, (3) administering zeolite to an animal, alone or in combination with the other elements of the present invention, and (4) using the kits of the present invention for improving stool quality comprising a document, digital storage media, optical storage media, audio presentation, or visual display containing the information or instructions.

24. The means of claim 23 selected from the group consisting of a displayed web site, brochure, product label, package insert, advertisement, or visual display.

25. A method for manufacturing a composition for animal consumption comprising admixing a stool quality improving amount of zeolite and at least one ingredient suitable for consumption by ah animal.
